(19) United States
(12) Patent Application Publication
(10) Pub. No.: US 2010/0087765 A1
(43) Pub. Date: Apr. 8, 2010

(54) Build-a-boot

(76) Inventor: Jeffrey W. Gainey, Peachtree City, GA (US)

Correspondence Address: Jeffrey W. Gainey, 417 B Dividend Drive, Peachtree City, GA, 30269, US

(21) Appl. No.: 12/287,136

(22) Filed: Oct. 7, 2008

(51) Int. Cl. A61F 5/00

(52) U.S. Cl. 602/23

(57) ABSTRACT

A heel supporting and protective boot for bed-ridden patients who may otherwise develop pressure sores on their heel. The body of the boot is comprised of a fiber and angled foam combination heretofore not used. The boot additionally has sewn tube holes on both sides allowing for use of medical tubing such as with a sequential compression pump. The inner portion material of the boot is a soft liner fabric which is antibacterial and absorbs moisture. The outer portion material of the boot is a fabric with fluid resistance. Further, the boot has a soft fiber-filled flap covering the shin portion of the leg to prevent pressure on the dorsal side of the lower leg. The boot additionally has a foot-position strap sewn and placed to the bottom of the boot running along both sides of the boot and attaching at the top of both sides of the upper boot to assist with the prevention of foot drop. Said invention further uses an opening at the heel which allows the heel to remain exposed to air as a result of proper elevation and cushioning created by the unique combination of fiber and angled foam in the body of said device. The elements incorporated address important factors in preventing or allowing healing of heel sores, or enhancing the curing of existing ulcers while minimizing chances of foot drop and minimizing chances of new pressure points. The fiber/foam combination, dual fabric content, shin flap, tubing holes, foot position strap and open heel design all work together to ensure proper healing and prevention of decubitis ulcers.

### BACKGROUND OF INVENTION

1. Field of Invention

This invention relates to the prevention or cure of heel sores for bed patients. The product is a medical boot that utilizes a unique combination of angled foam and fiber. The boot includes several other features that enhance the healing and prevention of heel sores while ensuring no other medical problems are created for the patient.

2. Background of the Prior Art

Heretofore several different foot and leg devices have been proposed for the cure or prevention of heel sores. These sores are known more specifically as decubitus ulcers, and generally form by frictional irritation between the heel of a bed ridden patient and the bed linens or mattress. Lying in a particular position for a prolonged period produces external pressures on skin and tissues covering rigid or bony body parts, such as the heel. This pressure compromises the blood supply to those rigid or bony areas which, after a sustained period, results in “bed sores.”

The following is a listing of patents issued for products intended to relieve heel pressure and thus resolve the problem of bed sores. Next to each listing is a brief description of the mechanics or method of their product.

U.S. Pat. No. 4,076,022 Therapeutic foot and leg protector—contains a rigid outer shell having a soft, protective liner. The protector is perforated in the heel portion for air circulation.
U.S. Pat. No. 6,256,804 Body Supports and protectors—The pillows and supports are made for supporting various body parts, namely neck, shoulder, torso, elbows, tail bone.
U.S. Pat. No. 5,328,445 Inflatable foot cushion—uses several, movable inflatable foot cushions and also involves the use of a shoe spoon and rigid base.
U.S. Pat. No. D411,758 Ornamental design for a heel protector. Product utilizes foam.
U.S. Pat. No. 5,367,789 Protective medical boot and orthotic splint—a soft medical boot with splint bar for adjusting angle of the foot. Includes rigid insole, rigid splint bar, hinge internal sac filled with air/water/gel, and rubberized laminated sole.
U.S. Pat. No. 5,449,339 Heel supporting protective boot for bed patients—foam with foot and leg supporting portions. Has friction reducing material and extra pad of foam to limit foot drop.

Although a number of devices have been developed for supporting and protecting the heels of bed patients, these devices have been subject to a number of difficulties and disadvantages which include the tendency to come loose, fall off of become disengaged from the patient as a result of patient movement and activity. Some products, that use foam only, can create additional pressure points. Some products that use fiber only can provide some cushioning but cannot sufficiently elevate the heel. These products do not off-load pressure from the heel, they only reduce heel pressure. These other products also have not heretofore allowed for the medical tubing of Sequential Compression Pumps to be used simultaneously. In addition, many prior art devices do not positively relieve and reduce pressure and friction between the heel or joint of the patient and the bedding. In addition, these devices allow the foot to drop below 90 degrees causing foot contracture and decreasing blood flow. Most of these devices can become soiled due to patient incontinency. In addition, many devices are hot and uncomfortable to wear and cause a build up of high temperatures. These conditions enhance the formation of decubitus ulcers. Many prior art products actually create high pressure contact points against the patient's body at which point sores often develop. In addition, these devices allow for internal/external rotation of the leg causing other joint and hip problems. Many of these devices are designed from foam alone that actually can harbor bacteria.

### OBJECTS AND SUMMARY OF THE INVENTION

It is an object of the present invention to provide a new and improved heel boot for bed patients which eliminates many of the disadvantages and problems encountered with prior art devices.

It is another object of the present invention to provide a new and improved heel supporting boot of the character described which is especially useful in the prevention or cure of bed sores on the heel.

Another object of the present invention is to provide a new and improved heel supporting boot which eliminates friction and pressure contact between the patient heel and bedding.

Another object of the invention is to provide an improved method of elevating the heel without hyper-extending the knee while facilitating the exposure of the heel to the circulation of air.

Another object of the invention is to utilize sewn holes in each side so that Sequential Compression Pump or other necessary medical tubing can be used with the product.

Another object of the invention is to utilize a combination of fiber and angled foam so that several goals of healing/preventing decubitis ulcers are met.

Another object of the invention is to provide a new product that does not allow for bacteria to grow inside the boot.

Another object of the invention is to provide a new product that utilizes moisture absorbing fabric on the interior while utilizes moisture repelling fabric on the exterior.

Another object of the invention is to provide a new method of strapping with the purpose of preventing foot drop of bed ridden patients.

Another object of the invention is to utilize a material that will ensure necessary air ventilation without heat build up.

Another object of the invention is to utilize the flat design shape of the foam to discourage internal/external rotation of the leg thus avoiding knee and hip joint problems.

Another object of the invention is to use a soft shin flap across the top of the leg to ensure that new pressure points are not created.

Another object of this invention is to ensure ease for the caregiver in putting on and taking off the device without ensuring ease of removal inadvertently.

Another object of the invention is to provide a better product that will not soil due to patient incontinence.

Another object of the invention is to ensure that there are no pressure points within the boot while maintaining an elevation of the heel.

Another object of the invention is to provide care for patients regardless of size or weight.

Another object of the invention is to provide a product which is economical to manufacture and simple to utilize while accomplishing an important goal.

The foregoing and other objects and advantages of the present invention are accomplished in an illustrated embodiment by way of representation and not limitation, which embodiment comprises a new and improved heel supporting boot for bed ridden patients. The boot has a unique combination of foam and fiber. The foam is either one or two pieces that are used singly or combined to provide a flat, wide base and an angled or sloped upper piece. The flat, wide base is utilized to discourage internal/external rotation of the patient's leg. The angled or sloped top or upper piece is utilized to ensure the patient's heel remains elevated and exposed to air without hyper-extending the knee joint. By elevating the heel, pressure is offloaded, not just reduced. The fiber works with the foam to eliminate or reduce any pressure points created by foam alone. The foam/fiber combination extends under the ankle and terminates at a point above the patients heel so the product is out of contact with the heel. The body includes a pair of sides designed to support the patient's lower leg and foot and the leg support fiber/foam portion extends between the sides to support the patient's leg with the heel elevated well above and out of contact with the bedding. The heel is completely elevated and thus exposed to air circulation through an opening in the product at the heel.

The boot also has tube holes sewn in each side that allow for the use of medical tubing at the same time the boot is used. The type of medical tubing typically utilized would be a Sequential Compression Pump.

The boot additionally uses dual types of fabric for the covering. The outer fabric repels fluid that may come into contact with the boot. The inner fabric absorbs fluids, like perspiration, and also is antibacterial.

The boot is made with a soft shin flap that ensures additional pressure points won't be created on the top of the patient's leg at the bony portions of the shin. This flap also helps with keeping the foot positioned in the boot by anchoring the foot. Additionally, the foot is prevented from rotating within the boot

Finally, the boot also uses a positioning strap which keeps the foot at a 90 degree angle and eliminates foot drop. By eliminating foot drop, essential blood flow is enhanced and heel cord tightening is eliminated. A 90 degree angle is the ultimate position which allows blood circulation

Still further objects and advantages of the invention will be readily apparent to those skilled in the art to which the invention pertains upon reference to the following detailed description.

### BRIEF DESCRIPTION OF THE DRAWINGS

For a better understanding of the invention, reference should be had to the following detailed description taken in conjunction with the drawings, in which:

FIG. 1 is top view of the foam wedge

FIG. 2 is a bottom view of the foam wedge

FIG. 3 is a side view of the foam wedge

FIG. 4 is a top view of the entire boot to show the positioning of fiber

FIG. 5 is back view of the boot and its wide foam bottom

FIG. 6 is top view of the boot and its soft shin flap

FIG. 7 is a side view of the boot to illustrate its positioning straps and sewn tube holes

FIG. 8 is an inside open view of the boot

FIG. 9 is an outside view of the side of the boot

### DESCRIPTION OF THE PREFERRED EMBODIMENT

Referring now more particularly to the drawings herein is illustrated a new and improved heel boot that utilizes several features combined to create a new and unique product.

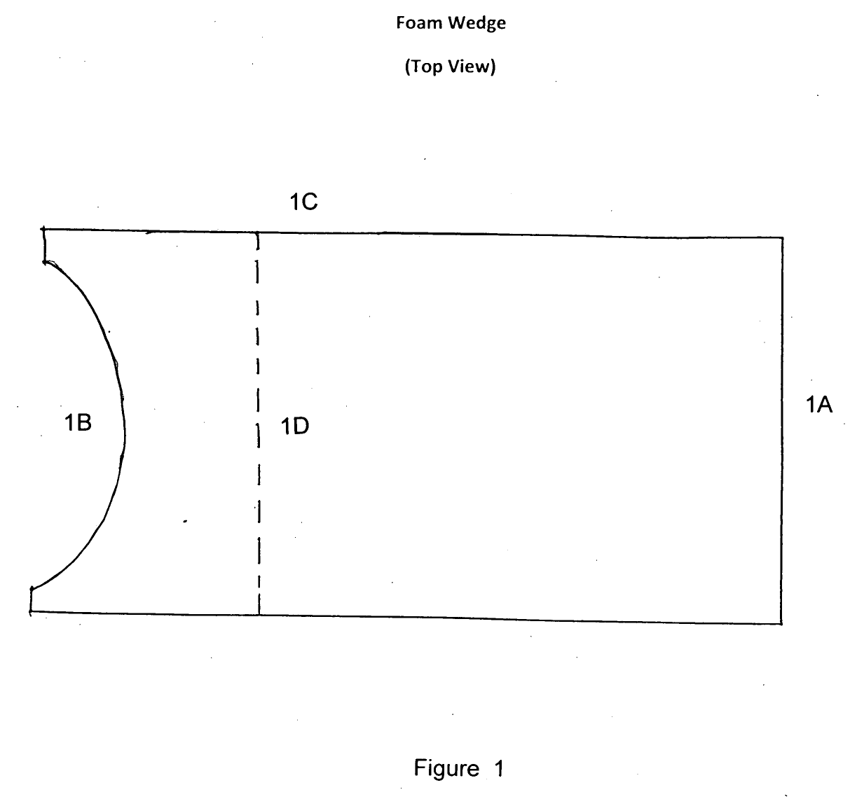

FIG. 1 is a top view of the foam wedge. This wedge of foam is used as the base of each boot. It is wide enough (FIG. 1A) to extend to the right and left side of each patient's lower leg. Its length (FIG. 1C) extends from the patients mid calf to their lower calf just above the ankle at 1B. At the portion which extends to the ankle (1B) there is a circular cutout that ensures the heel will remain offloaded, elevated and exposed to air. The width of the boot is formulated to ensure stability of the boot. The foam wedge slopes from 1A to 1D at an approximately 30 degree angle until it reaches 1D. At 1D the slope ends and the top of the foam wedge is flat.

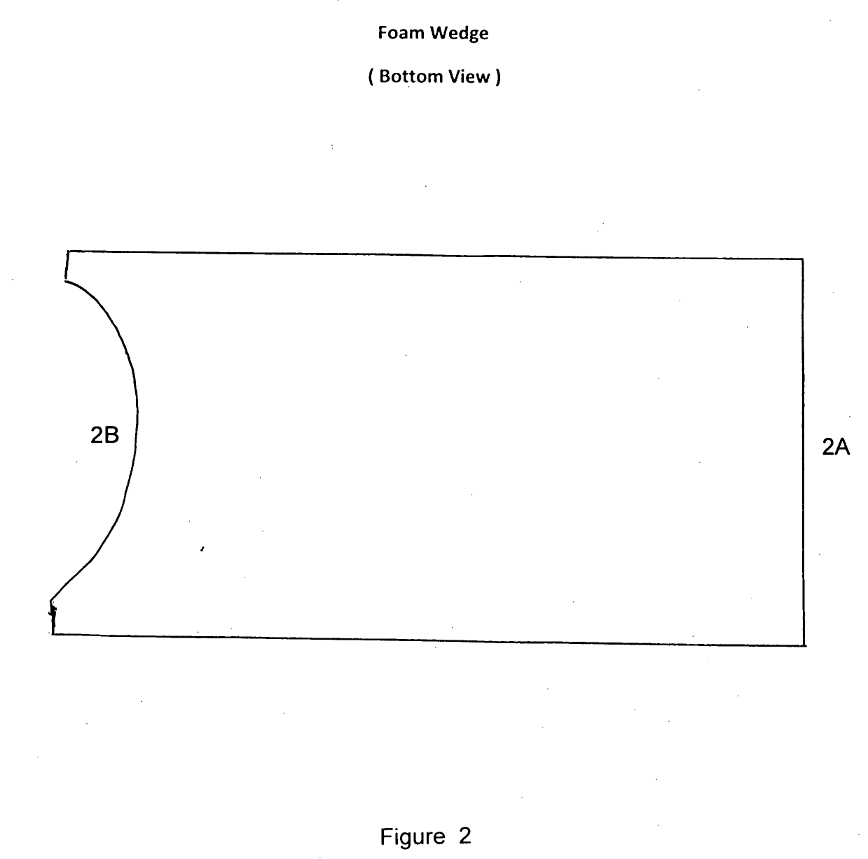

FIG. 2 is a bottom view of the foam wedge and illustrates that the bottom of this foam wedge is flat on the portion of the boot that would come into contact with the patients bedding. The flat, wide makeup of this wedge inhibits internal or external rotation of the foot within the boot. 2B illustrates the cutout referred to for 1B. 2A illustrates the width which extends from one side of the patients lower leg to the other side.

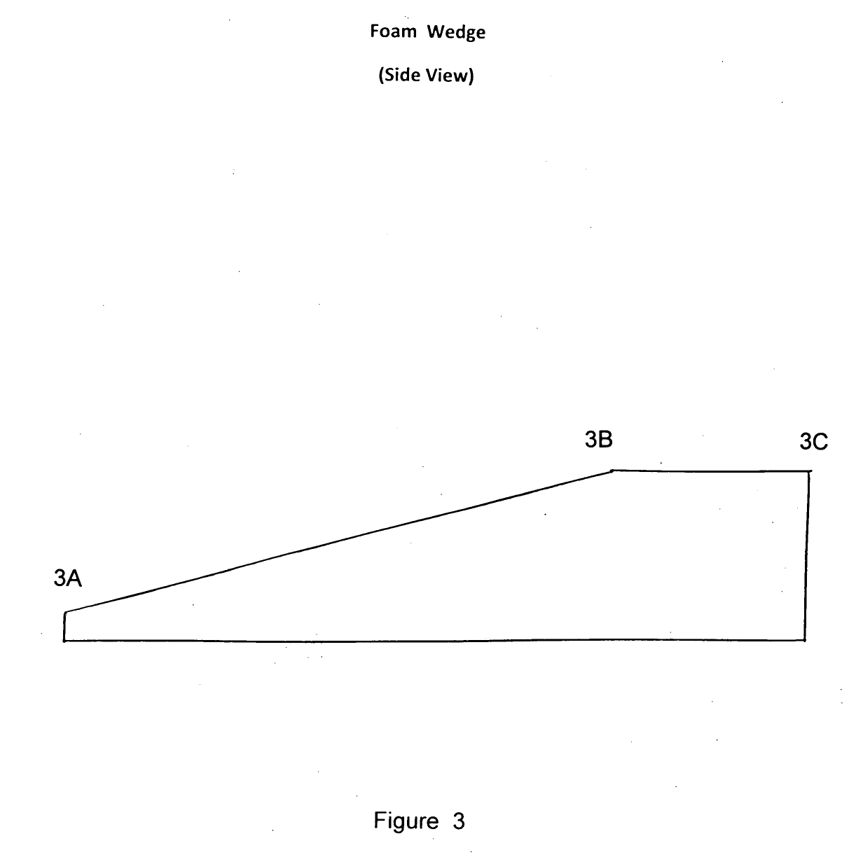

FIG. 3 is a side view of the foam wedge. This illustration better shows the 30 degree slope from 3A to 3B at which point the slope ends and the top of the wedge is flat. From 3B to 3C the top and bottom of the foam wedge are parallel. The point at 3c terminates at a point that would lie just beneath the patient's ankle. This foam wedge ensures that the patient's ankle is offloaded and elevated above the bedding to reduce pressure, that the heel remains exposed to air and that maximum blood flow is achieved.

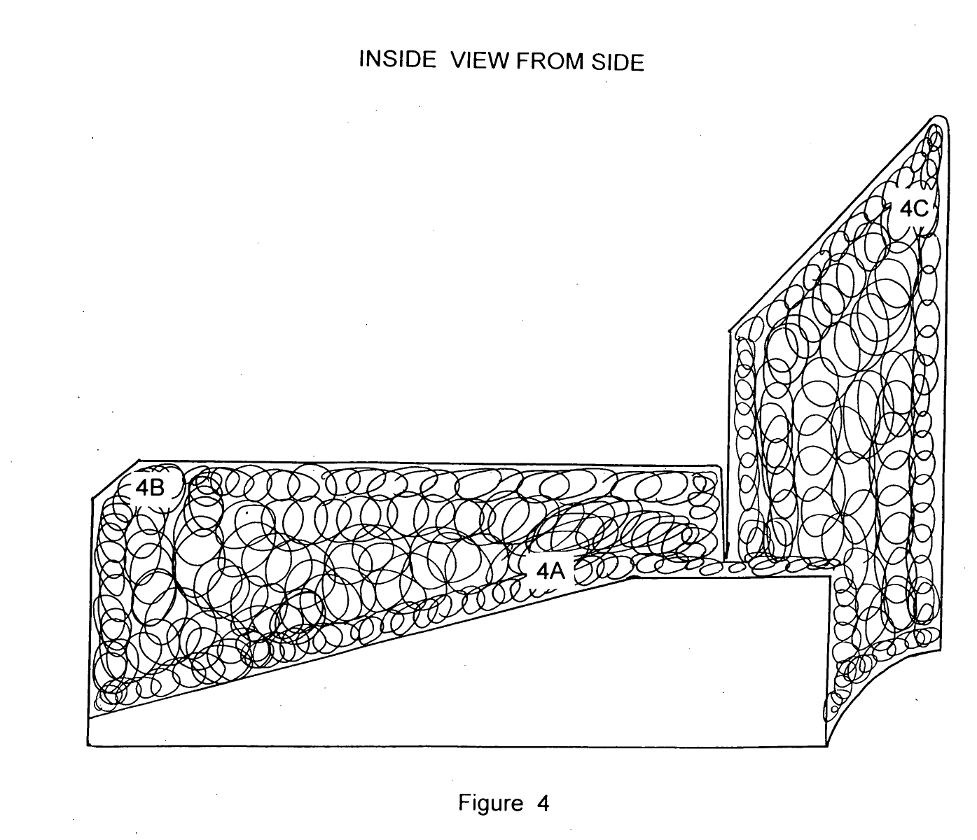

FIG. 4 is an internal view of the boot. The foam/fiber combination is unique. The base of the boot consists of the fiber wedge, featured in FIGS. 1-3, and fiber is placed strategically at 4A, 4B, 4C and the space between these three points. The fiber lies atop the upper foam wedge (FIG. 4A), on each of the side walls (FIG. 4B) and at the foot portion (FIG. 4C) of the boot. The purpose of the fiber is to cushion the bony areas of the lower leg and prevent pressure sores that can be caused by the use of foam only.

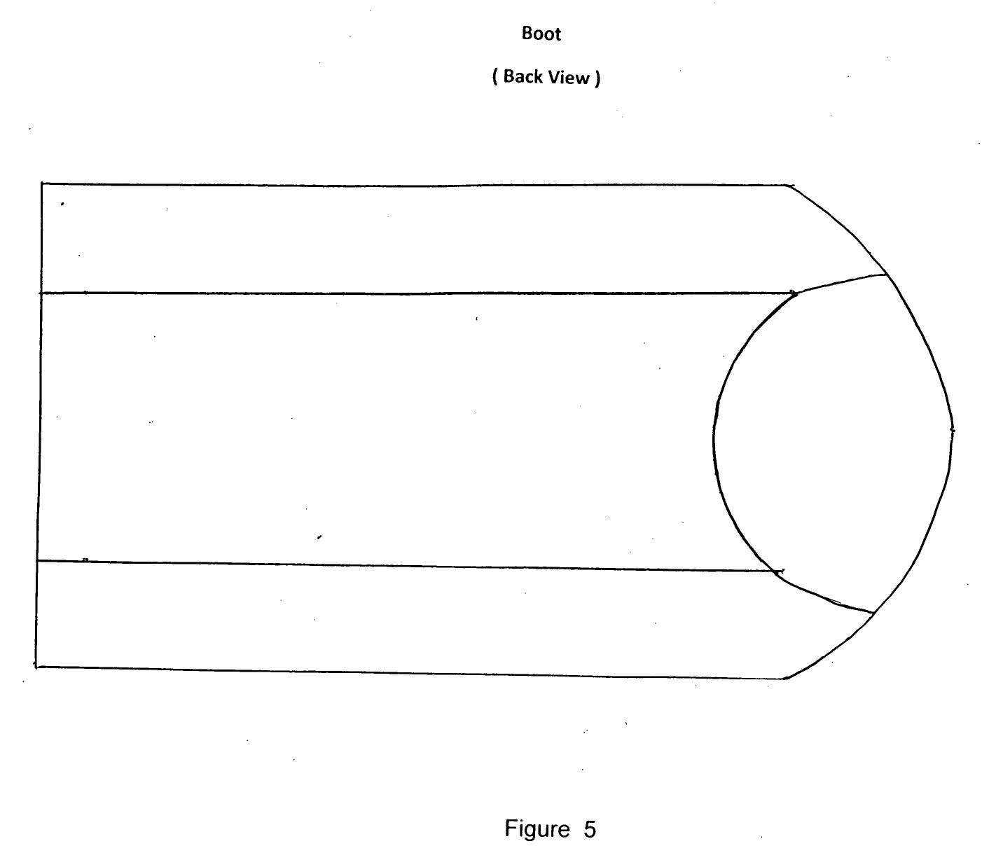

FIG. 5 is a bottom or back view of the boot showing the base of the boot and each of the side walls. The base of the boot is the portion that would have direct contact with the patient's bedding. The base is made from the foam wedge and the side walls are made with the fiber filling. The base of the boot is a width that ensures internal and external rotation of the leg is limited.

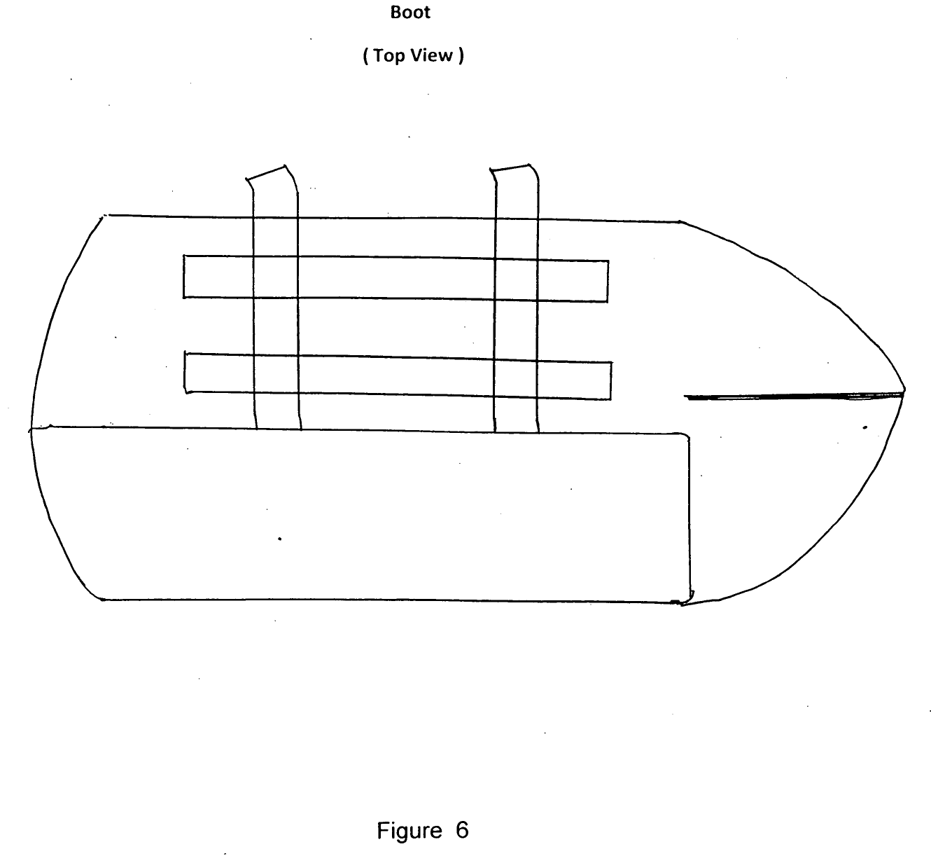

FIG. 6 is a top view of a closed boot. The boot is closed by the use of a soft shin flap. The flap crosses over itself and is secured together by the use of hook and loop straps. The right and left portion of the side wall of said boot extend into a more flexible portion that is considered the flap. Each flap is filled with fiber to ensure cushioning. Each flap is brought over the top of the patient's shin and once in place, said flaps are held secure with hook and loop straps. The cushioned flaps ensure no new pressure points are created and are important to keep the foot positioned within the boot. These flaps keep the foot anchored and prevent the foot from rotating within the boot.

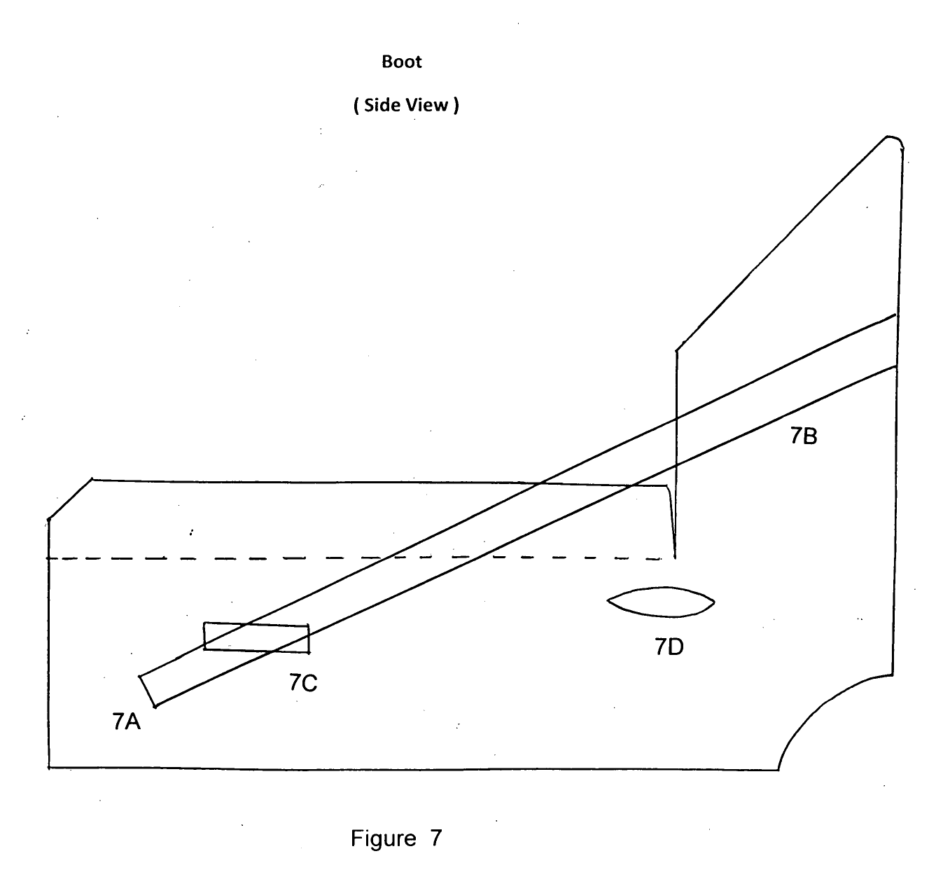

FIG. 7 is a right side view of the boot and is used to illustrate the positioning straps. These straps keep the foot at a 90 degree angle which is the ultimate position to allow continued blood circulation and prevent foot drop. Foot drop can result in heel cord tightening. The strap begins at a point (FIG. 7B) under the upper foot portion of the boot and ends at point (FIG. 7A) along the calf of the patient. The end of the strap (FIG. 7A) is adhered to the boot by the use of a sewn piece of hook at 7C. An identical strap would run from the upper foot to upper shin on the left side of the boot also. The straps on each side of the boot work together to maintain the patient's foot at a 90 degree angle.

FIG. 7 is also used to illustrate the sewn hole (FIG. 7D) for medical tubing. The main medical tubing that would typically be used in conjunction with a pressure relief boot would be a Sequential Compression Pump. Many patients that would require such a boot as illustrated herein would also need the use of medical tubing and these sewn holes allow for the use of said tubing while still using the pressure relief of the boot.

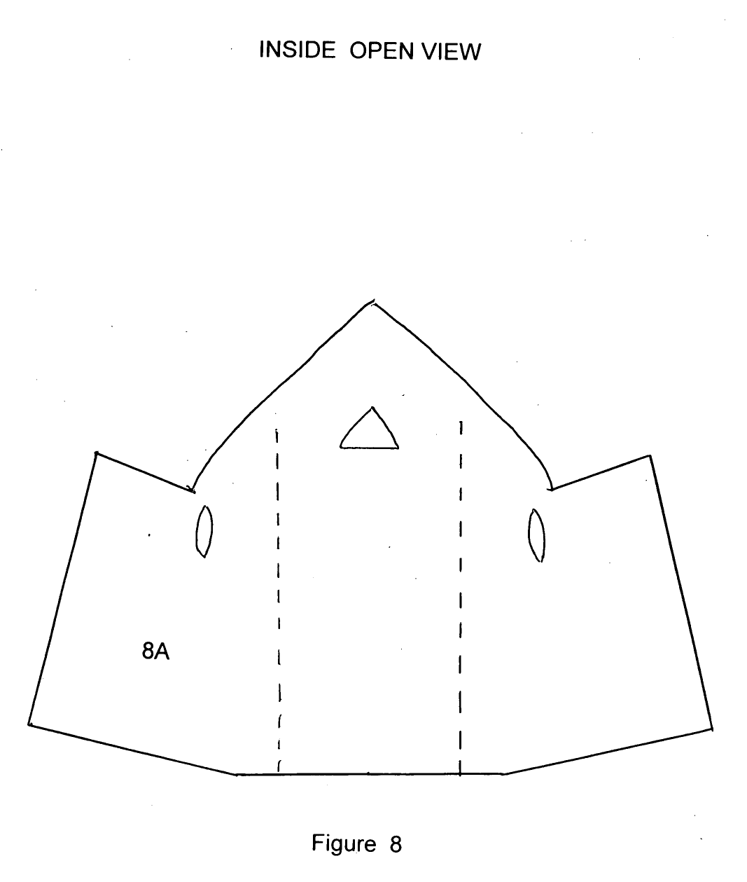

FIG. 8 is an inside view of an open boot. The inner portion of the boot (FIG. 8A) utilizes a fabric that has two features. The first feature is that this fabric is antibacterial meaning it will not allow bacteria to grow and creates an environment hostile to further growth of bacteria. The second feature is that this fabric absorbs moisture that may be produced by the patient.

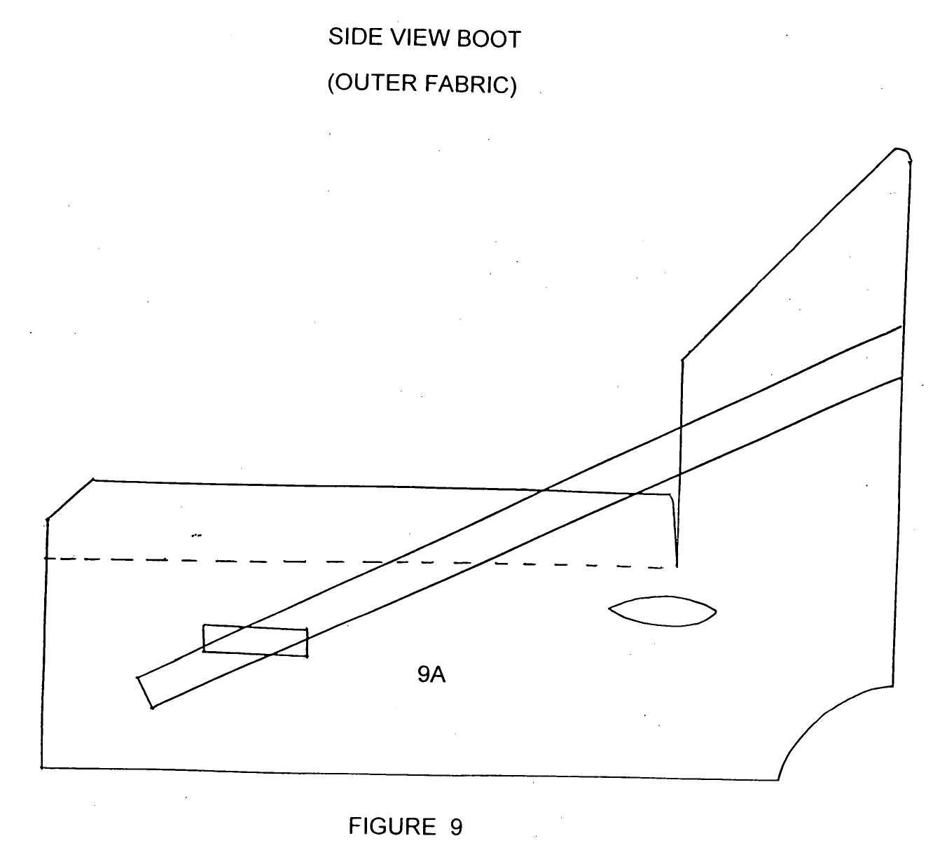

FIG. 9 is an outside view of the boot. The outer portion of the boot utilizes a fabric that repels fluids.

While the invention has been illustrated and described in detail in the drawings and foregoing description, the same is to be considered as illustrative and not restrictive in character, it being understood that only the preferred embodiment has been shown and described an that all changes and modifications that come within the spirit of the invention are desired to be protected.

What is claimed and desired to be secured is:

1. A heel elevating, cushioned foot boot for bed patients that uses a unique combination of angled foam and fiber.

2. The foot boot of claim 1, wherein medical tube holes are sewn into each side wall of the boot.

3. The foot boot of claim 1 and 2, wherein the inner fabric utilized deters bacteria growth and absorbs moisture.

4. The foot boot of claim 1 and 2, wherein the outer fabric utilized repels moisture.

5. The foot boot of claim 1 and 2, wherein a new method of strapping is utilized for the purpose of preventing foot drop of bed patients.

6. The foot boot of claim 1 and 2 that uses a soft shin flap across the top of the leg that ensures new pressure points are not created and maintains the foot position within the boot.
